(19) United States
(12) Patent Application Publication
(10) Pub. No.: US 2013/0085109 A1
(43) Pub. Date: Apr. 4, 2013

(54) METHODS OF CARDIOVASCULAR PATIENT TREATMENT USING SUBSTANCES SUFFICIENT TO REDUCE ACTIN DEPOLYMERIZATION

(71) Applicants: Ghassan S. Kassab, Zionsville, IN (US); Zhen-Du Zhang, Carmel, IN (US)

(72) Inventors: Ghassan S. Kassab, Zionsville, IN (US); Zhen-Du Zhang, Carmel, IN (US)

(21) Appl. No.: 13/683,920

(22) Filed: Nov. 21, 2012

Related U.S. Application Data
(63) Continuation-in-part of application No. 12/248,507, filed on Oct. 9, 2008.
(60) Provisional application No. 61/562,216, filed on Nov. 21, 2011.

(51) Int. Cl. A61K 38/15; A61K 31/336

(52) U.S. Cl. CPC A61K 38/15; A61K 31/336
USPC 514/13.5; 514/21.1; 514/475; 514/16.4

(57) ABSTRACT

Methods of cardiovascular patient treatment using substances sufficient to reduce actin depolymerization. In an exemplary embodiment of a method of treating a patient of the present disclosure, the method comprises the step of administering a therapeutically effective dose of a substance that reduces actin depolymerization within a vasculature of a patient to treat a cardiovascular condition of the patient. In at least one embodiment, the substance the substance is selected from the group consisting of jasplakinolide, Jasplaskinolide V, and Amphidinolide H.

### PRIORITY

The present application (a) is related to, and claims the priority benefit of, U.S. Provisional Patent Application Ser. No. 61/562,216 filed Nov. 21, 2011, and (b) is related to, claims the priority benefit of, and is a continuation-in-part application of, U.S. Nonprovisional patent application Ser. No. 12/248,507, filed Oct. 9, 2008, which is related to, claims the priority benefit of, and is a divisional application of, U.S. Nonprovisional patent application Ser. No. 11/659,876, filed Feb. 9, 2007, now abandoned, which is related to, claims the priority benefit of, and is a U.S. National Stage Application of PCT Application No. PCT/US2005/028818, filed Aug. 11, 2005, which is related to, and claims the priority benefit of, U.S. Provisional Patent Application Ser. No. 60/601,092, filed Aug. 11, 2004. The contents of each of these applications are hereby incorporated by reference in their entirety into this disclo sure.

### RESEARCH SUPPORT

This invention was made with government support under Grant No. R01 HL084529 awarded by National Institutes of Health—National Heart, Lung and Blood Institute. The government has certain rights in the invention.

### BACKGROUND

Nitric oxide (NO) is a compound that is produced by healthy endothelial cells, such as those that line human blood vessels, and is associated with relaxation and dilation of blood vessels. Nitric oxide acts as a signaling molecule in the cardiovascular system. A signaling molecule is a molecule that produces or induces the production of another substance, called a second messenger. The second messenger then brings about some physiologic effect.

When nitric oxide (the signaling molecule) enters a cell, it activates an enzyme called guanylate cyclase, which then causes production of cyclic GMP (the second messenger). The cyclic GMP then causes relaxation and dilation of the blood vessels. In addition to relaxing and dilating blood vessels, nitric oxide also prevent coronary artery disease and strokes by preventing platelets and white blood cells from sticking to the vessel wall. Nitric oxide may also, under certain conditions, reduce the presence of free radicals, which can cause your vessels to age rapidly. Also, nitric oxide suppresses abnormal growth of vascular smooth muscle cells as is known to occur in certain types of atherosclerosis and during reocclusion following balloon angioplasty procedures.

Hypercholesterolemia reduces nitric oxide bioavailability, which in turn results in reduced endothelium-dependent vascular relaxation, and also induces the expression of vascular adhesion molecules and infiltration of inflammatory cells. It has been reported that gene therapy with Nitric Oxide synthase in hypercholesterolemic rabbits substantially reverses the deficit in vascular relaxation exhibited by those animals.

Transient near wall blood flow reversal (negative wall shear stress (“WSS”)) occurs during diastole with each heart beat in peripheral vessels. More prominent retrograde arterial blood flows have been reported in diseased conditions, especially in heart failure. In general, slow moving blood flow and changes in flow direction cause endothelial dysfunction and disease. In contrast, vessel regions that are exposed to steady blood flow are not as prone to atherosclerosis.

Although the cause of endothelial cell (“EC”) dysfunction due to change of WSS may be complex, increased evidence suggests that excessive production of reactive oxygen species (“ROS”) may play an important role in this process. It has been shown that NADPH oxidase has a directional response to shear stress.

The cytoskeleton plays an important role in maintaining cellular structural integrity through the 3D filament network. The cytoskeleton may also mediate the signal transduction between surface membrane and intracellular target. The disruption of the actin network has been shown to reduce angiotensin II-induced Ca²⁺ release in smooth muscle cells or angiotensin II induced ERK1/2 activity. It has been confirmed that fluid shear stress exerts significant influences on the endothelial cells in cell orientation and morphology, cytoskeleton organization, signal transduction and growth factor mediation, and gene expression. Endothelial cells can sense differences in the temporal and/or spatial characteristics of flow and translate these biochemical stimuli into biological responses.

It has been suggested that the endothelial cell membrane integrin may serve as sensor for WSS. Several actin binding proteins serve as platform for integrin-mediated signal transduction. In conjunction with external ligands, integrins activate signaling cascades that regulate the formation, turnover and linkage of actin filaments. One of the outcomes of this pathway may be the change of NADPH oxidase activity and superoxide production, and actin may transmit surface mechano-stimulation to the NADPH oxidase.

In vivo evidence for NADPH oxidase regulation by actin in endothelial cells is not currently present.

### BRIEF SUMMARY

In an exemplary embodiment of a method of treating a patient of the present disclosure, the method comprises the step of administering a therapeutically effective dose of a substance that reduces actin depolymerization within a vasculature of a patient to treat a cardiovascular condition of the patient. In at least one embodiment, the substance is selected from the group consisting of jasplakinolide, Jasplaskinolide V, and Amphidinolide H. In another embodiment, the cardiovascular condition comprises blood flow reversal. In yet another embodiment, the therapeutically effective dose of the substance does not inhibit the incidence of blood flow reversal.

In an exemplary embodiment of a method of treating a patient of the present disclosure, the cardiovascular condition comprises congestive heart failure. In an additional embodiment, the therapeutically effective dose is effective to prevent actin depolymerization with the vasculature of the patient. In yet an additional embodiment, the therapeutically effective dose of the substance also inhibits an effect of superoxide within the vasculature of the patient. In another embodiment, the therapeutically effective dose of the substance also inhibits superoxide production within the vasculature of the patient, wherein the superoxide production is induced due to the cardiovascular condition.

In an exemplary embodiment of a method of treating a patient of the present disclosure, the therapeutically effective dose of the substance also inhibits superoxide production by endothelial cells within the vasculature of the patient. In another embodiment, the therapeutically effective dose of the substance also inhibits NADPH oxidase activity within the vasculature of the patient. In yet another embodiment, the therapeutically effective dose of the substance also inhibits p47^{phox} phosphorylation within the vasculature of the patient. In an additional embodiment, the therapeutically effective dose of the substance also inhibits NADPH oxidase complex formation within the vasculature of the patient.

In an exemplary embodiment of a method of treating a patient of the present disclosure, the therapeutically effective dose of the substance treats an effect of blood flow reversal within the vasculature of the patient at a location of the blood flow reversal. In an additional embodiment, the therapeutically effective dose is less than 1.0 μM of the substance. In an additional embodiment, the therapeutically effective dose does not inhibit a naturally-occurring incidence of superoxide production within the patient, but does inhibit superoxide production by endothelial cells in connection with an incidence of blood flow reversal within the vasculature of the patient.

In an exemplary embodiment of a method of treating a patient of the present disclosure, the method comprises the step of administering a therapeutically effective dose of a substance selected from the group consisting of jasplakinolide, Jasplaskinolide V, and Amphidinolide H to a patient to treat a cardiovascular condition of the patient, wherein the cardiovascular condition includes at least an incidence of blood flow reversal within a vasculature of the patient. In another embodiment, the therapeutically effective dose of the substance also inhibits superoxide production by endothelial cells within the vasculature of the patient. In yet another embodiment, the therapeutically effective dose of the substance also inhibits NADPH oxidase activity within the vasculature of the patient.

In an exemplary embodiment of a method of treating a patient of the present disclosure, the method comprises the step of introducing a quantity of a substance within a portion of a cardiovascular system, the quantity sufficient to (a) reduce a rate of actin depolymerization resulting from blood flow reversal within the circulatory system, (b) inhibit superoxide production by endothelial cells within the vasculature of the patient, and (c) inhibit NADPH oxidase activity within the vasculature of the patient. In another embodiment, the substance is selected from the group consisting of jasplakinolide, Jasplaskinolide V, and Amphidinolide H.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIGS. 1A and 1B show an in vivo system, according to an exemplary embodiment of the present disclosure;

FIG. 1C shows blood flow recordings in connection with uses of the systems shown in FIGS. 1A and 1B, according to an exemplary embodiment of the present disclosure;

FIG. 2A includes a chart of relative superoxide production in the presence of certain compounds, according to an exemplary embodiment of the present disclosure;

FIG. 2B includes a chart of positive control data in connection with superoxide production, according to an exemplary embodiment of the present disclosure;

FIGS. 3A and 3B include charts showing the effects endothelial cell denudation on superoxide production with forward flow and reverse flow, respectively, according to exemplary embodiments of the present disclosure;

FIG. 4 includes a chart showing the effect of reverse flow on superoxide production, according to an exemplary embodiment of the present disclosure;

FIGS. 5A-5E show photographs of immunostained actin, according to exemplary embodiments of the present disclosure;

FIGS. 6A and 6B include charts showing the effects of reverse flow on NADPH oxidase activity, according to exemplary embodiments of the present disclosure;

FIG. 6C shows images of western blot samples, according to an exemplary embodiment of the present disclosure;

FIG. 7A includes a chart showing the level of phosphorylated p47^{phox} in vitro, according to an exemplary embodiment of the present disclosure;

FIG. 7B shows images of western blot samples in connection with FIG. 7A, according to an exemplary embodiment of the present disclosure;

FIG. 7C includes a chart showing levels of phosphorylated p47^{phox} under forward flow in vivo, according to an exemplary embodiment of the present disclosure;

FIG. 7D shows images of western blot samples in connection with FIG. 7A, according to an exemplary embodiment of the present disclosure;

FIG. 8 shows a graph showing production of nitric oxide metabolite versus flowrate (forward and reverse) in ex vivo porcine femoral artery preparations with and without tempol, according to an exemplary embodiment of the present disclosure; and

FIG. 9 shows micrographs obtained by laser confocal microscopy showing the concentrations of superoxide in carotid artery wall under control (forward flow), reverse flow (FR), and reverse flow plus apocynin (AC) conditions, according to exemplary embodiments of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

The present disclosure tests the hypothesis that actin depolymerization that occurs during flow reversal induces superoxide production in vascular endothelial cells mediated by NADPH oxidase and more specifically p47^{phox}. In view of the same, evidence is provided herein to confirm that reverse flow-induced superoxide production in endothelial cells via actin microfilament depolymerization potentiates NADPH oxidase activity both in vitro and in vivo.

Flow reversal, which is known to occur in CHF, is believed to increase in superoxide production and reduce nitric oxide levels in large vessels. In peripheral vessels, CHF will lead to reduction in flow and similar increase in superoxides. Accordingly, the present disclosure provides treatments for congestive heart failure (CHF) based on inhibition or reduction of superoxides and/or increasing nitric oxide.

In accordance with the present disclosure, there is provided a method for treating heart failure in a human or animal subject, such method comprising the step of administering to the subject, in an amount that is therapeutically effective to increase the concentration of nitric oxide in blood vessels of the heart, at least one substance selected from the group consisting of a) SOD mimics, b) NADPH oxidase inhibitors and c) substance that inhibits the effect of superoxide and/or reduces the amount of superoxide and/or increases the amount of nitric oxide present in the affected tissues (e.g., the subject's blood vessels and/or heart). For example, superoxide is produced in the cell through various pathways. These pathways are mitochondrial oxidase, xanthine oxidase (XO), uncoupled NO synthases, cytochrome P-450 enzymes, and NADPH oxidases. In addition, enzymes such as lipoxygenases may also generate superoxide. In particular, both the NADPH oxidase pathway and the XO pathway have been implicated in endothelial dysfunction in artherosclerosis and heart failure. Inhibitors of these pathways include, but are not limited to; NADPH Oxidase inhibitors such as apocynin and 4-(2-aminoethyl)-benzenesulfonyl fluoride (AEBSF); 2. Xanthine oxidase inhibitors such as allopurinol or oxypurinol and 3. other oxidase inhibitors, such as Diphenyleneiodonium (DPI) which inhibits NADPH oxidase, XO, nitric oxide synthase, cytochrome P-450 reductase, and mitochondrial oxidase. Further in accordance with the present disclosure, there are provided pharmaceutical preparations for the treatment of CHF, such preparations comprising a) at least one substance selected from the group consisting of i) SOD mimics, ii) NADPH oxidase inhibitors and iii) substances that inhibit superoxide or reduce the amount of superoxide and/or increase the amount of nitric oxide present in affected tissues (e.g., the subjects blood vessels and/or heart) in combination with b) at least one solvent, carrier, vehicle, medium, diluent or excipient agent.

The present disclosure provides methods for treating a cardiovascular disorders such as congestive heart failure (CHF) in human or animal subjects by administering to the subject a therapeutically effective amount of a substance that mimics the action of superoxide dismutase (SOD) (hereinafter referred to as “SOD mimics”) and/or any other substance that decreases production of, scavanges, blocks the effects of, reduces the amount of or otherwise inhibits superoxide and/or increases the concentration of nitric oxide.

There are two major classes of SOD mimics, those that contain metals and those that are metal-independent. The three metals contained in complexes normally studied are copper, iron, and manganese. Metal-independent SOD mimics are various nitroxides complexes. SOD mimics catalyze the dismutation of O.sub.2 to hydrogen peroxide (H.sub.2O.sub.2) and dioxygen (O.sub.2). Examples of SOD mimics useable in this invention include those described or disclosed in U.S. Pat. No. 6,180,620 (Salvemini); U.S. Pat. No. 6,214,817 (Riley et al.); U.S. Pat. No. 6,245,758 (Salvemini); U.S. Pat. No. 6,395,725 (Salvemini); U.S. Published Patent Application No. US2002/0072512 A1 (Salvemini); U.S. Published Patent Application No. US2002/0128248 A1 (Salvemini); U.S. Published Patent Application No. US2004/0132706 A1 (Salvemini) and Salvemini, D., et el., Therapeutic Potential of Superoxide Dismutase Mimetics as Therapeutic Agents in Critical Care Medicine, Crit. Care Med 2003, Vol. 31, Vol. 1, each of the aforementioned patents and patent applications being expressly incorporated herein by reference.

The SOD mimics used in the present invention may have the general formula:

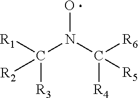

wherein R₁, R₂, R₅ and R₆ are each independently C₁-C₂₀ alkyl, C₂-C₂₀ alkenyl or C₂-C₂₀ alkynyl; or R₁ and R₂ and/or R₅ and R₆ combine, with the linking atom, to form a C₃-C₁₂ cyclic ring; and

wherein R₃ and R₄ may combine, including the nitrogen atom (N), to form a heterocyclic 5-7 member ring structure, wherein (i) at least one ring methylene group is replaced by a heteroatom selected from NR, O and S or an oxo (C═O) group, where R is selected from H, lower alkyl, lower alkenyl, lower alkynyl; and (ii) at least one methylene group hydrogen is substituted with a group selected from C₁-C₂₀ alkyl, C₂-C₂₀ alkenyl, C₂-C₂₀ alkynyl, —OH, —O—CO—R₇, —O—R₇, —S—R₇ and —NR₈R₉, where R₇ is selected from C₁-C₂₀ alkyl, C₂-C₂₀ alkenyl, C₂-C₂₀ alkynyl, aralkyl and glycosyl; R₈ and R₉ are selected independently from H, C₁-C₂₀ alkyl, C₂-C₂₀ alkenyl, C₂-C₂₀ alkynyl, aryl, aralkyl, glycosyl and —CO—R₁₀, where R₁₀ is lower alkyl or cycloalkyl.

One commercially available SOD mimic substance that is useable in the present invention is tempol (4-hydroxy-2,2,6,6-tetramethyl-4-piperidine-N-oxyl). Tempol is commercially available from Sigma Chemical, St. Louis, Mo. To treat CHF in a human patient, tempol may be administered at a dose of from about 2 to about 200 mg/kg or from about 5 to about 55 mg/kg, i.v., or by other routes of administration at doses that are higher or lower than 5 to about 55 mg/kg. It will be appreciated that any effective dosages and/or routes of administration and/or dosing schedules may be employed.

The potential utility of tempol and other SOD mimics in the treatment of CHF has been established based on laboratory testing, as described in Example 1 below:

### Example 1

### The Effects of Tempol On Superoxide Production During Flow Reversal

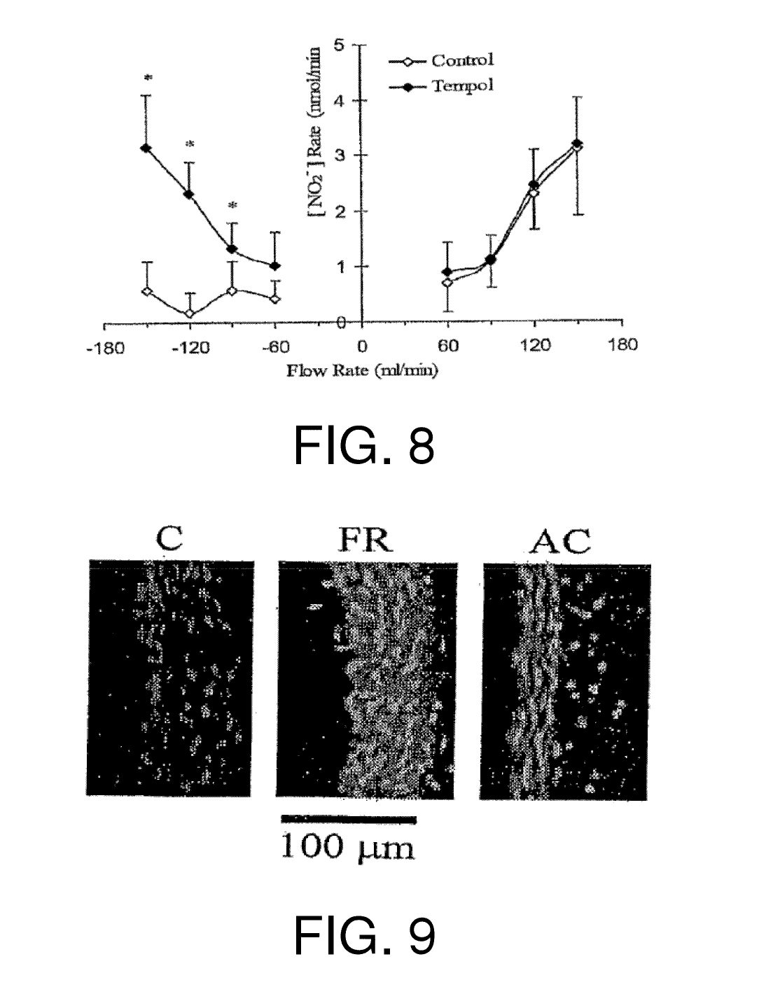

A purfusate was passed through an in vitro porcine femoral artery preparation in the forward and reverse flow directions and the levels of NO metabolite (nitrite) were measured at various flowrates in the forward and reverse directions. Thereafter, tempol was added to the perfusate and the levels of NO metabolite (nitrite) were again measured at various flowrates in the forward and reverse directions. The addition of tempol to the perfusate did not cause a statistically significant increase in NO production during forward flow. However, the addition of tempol did significantly increase NO production during reverse flow. These data (mean.+−.SD) are shown graphically in FIG. 8. The asterisk denotes statistical significance by ANOVA analysis between groups at the respective flows (P<0.05).

Another category of compounds that may be used in accordance with the present invention are oxidase blockers such as NADPH oxidase inhibitors. One such NADPH oxidase inhibitor is apocynin (4-hydroxy-3-methoxy-acetophenone). Other NADPH Oxidase inhibitors include, for example, 4-(2-aminoethyl)-benzenesulfonyl fluoride (AEBSF) and Diphenyleneiodonium (DPI). The potential utility of apocynin and other NADPH Oxidase inhibitors in the treatment of CHF has been established based on laboratory testing, as described in Example 2 below:

### Example 2

### The Effect of Apocynin on Superoxide Concentration in Arterial Wall During Flow Reversal

The carotid arteries of animals were removed and examined by laser confocal microscopy excitation (514 nm; emission, 605 nm; Objective, 40x. C.3.a. Visualization of NO and O₂-Using Fluorescence) following perfusion under conditions of: Control (C), Flow Reversal (FR), and Apocynin+Flow Reversal (AC). The apocynin was administered in drinking water (1 mM) to an animal concurrently with carotid flow reversal. Dihydroethidine (DHE), a fluorescent dye, was used to indicate superoxide in arterial wall. As seen in FIG. 9, the animal that received Apocynin+Flow Reversal clearly had less superoxide present in the artery wall than the animal that received Flow Reversal without apocynin. Thus, the administration of apocynin reduced the amount of superoxide present under reverse flow conditions.

The dosages of apocynin useable to treat CHF are in the range of from about 2 mg/kg to about 200 mg/kg and more preferably in the range of from about 4 mg/kg to about 40 mg/kg, i.v., or by other routes of administration and/or at other dosages. It will be appreciated that any effective dosages and/or routes of administration and/or dosing schedules may be employed.

Although specific examples are provided above with respect to only tempol and apocynin, such examples also generally demonstrate the utility of any therapy that inhibits superoxide or reduces the amount of superoxide and/or increases the amount of nitric oxide present in affected tissues (e.g., the subject's blood vessels and/or heart).

### Additional Experiments

A total of twenty domestic male swine (40-55 kg) were used to test the general hypothesis referenced herein, namely that actin depolymerization that occurs during flow reversal induces superoxide production in vascular endothelial cells mediated by NADPH oxidase and more specifically p47^{phox}. The protocols for animal use were approved by the Institutional Animal Care and Use Committee of Indiana University Purdue University Indianapolis. In all experiments, the animals were fasted overnight and given an intra-muscular injection of TKX (Telazol 10 mg/kg, ketamine 5 mg/kg, and xylazine 5 mg/kg) as an initial anesthetic. The animals were intubated and mechanically ventilated with oxygen (100%) and isoflurane (1-2%). Vital signs (such as heart rate, respiratory rate, SpO₂, ETCO₂), body temperature, ECG and blood pressure were monitored and maintained within the physiological range during the experimental duration.

In each animal, an mid-incision was made on the neck to expose the carotid arteries without complete separation from the surrounding tissues. To perform various tests, including but not limited to those specifically recited below, a novel in vivo system 100 of the present disclosure was developed and used.

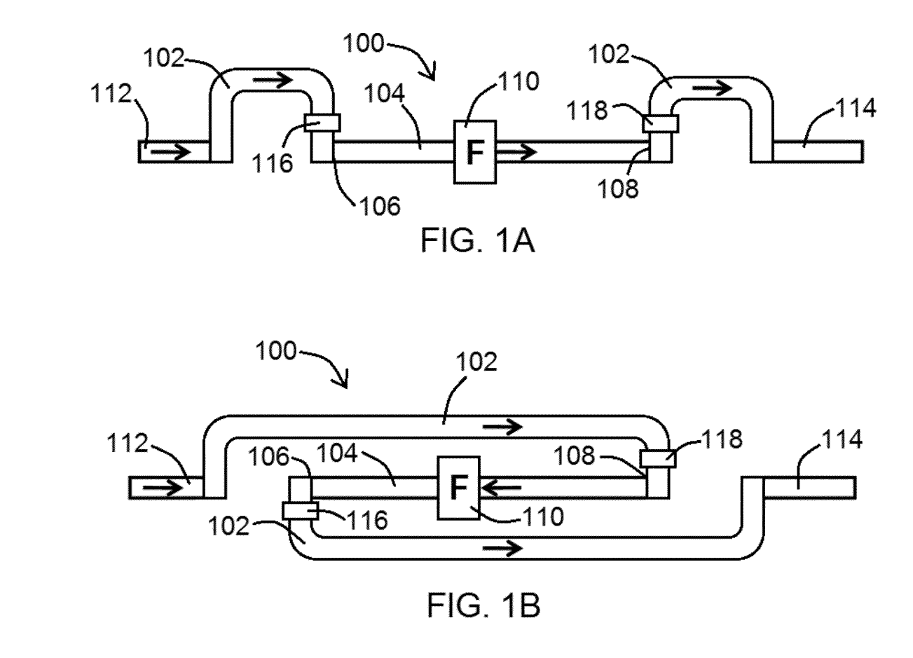

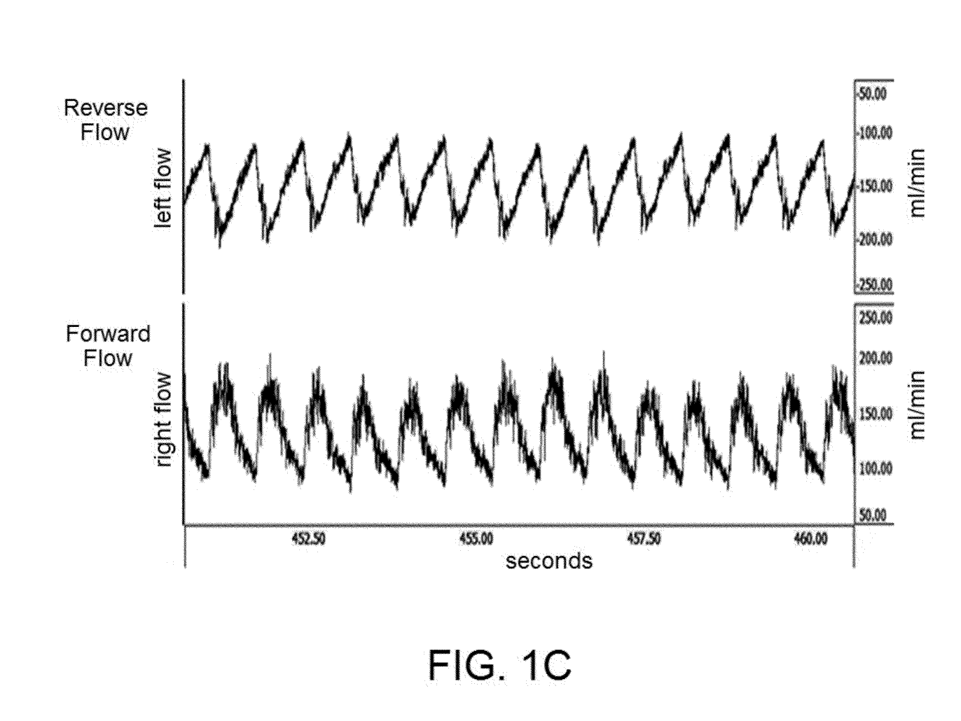

To create a full reverse flow, and as part of an exemplary system 100 of the present disclosure, two tubes 102, such as two pieces of silicone tubing, were inserted into the two respective ends of the isolated vessel segment 104 as shown in FIGS. 1A and 1B. In at least one embodiment, tubes 102 are configured to fit within a portion of isolated vessel segment 104, and in at least another embodiment, tubes 102 are configured so surround the ends of vessel segment and are held in place with a connector (not shown). FIGS. 1A and 1B shows illustrations of exemplary in vivo carotid artery blood flow setups (systems 100) of the present disclosure. Forward (FIG. 1A) or reverse (FIG. 1B) flows were created by insertion of silicone tubes 102 connecting proximal and distal incisions on carotid artery, namely whereby one tube 102 is connected to a proximal end 106 of vessel segment 104, and whereby another tube 102 is connected to a distal end 108 of vessel segment 104. In FIG. 1A, the proximal tube 102 is connected to proximal end 106 of vessel segment 104 and the distal tube 102 is connected to distal end 108 of vessel segment 104. In FIG. 1B, to facilitate reverse flow, the proximal tube 102 is connected to distal end 108 of vessel segment 104 and the distal tube 102 is connected to proximal end 106 of vessel segment 104. The tubes 102 used in forward (FIG. 1A) and reversed (FIG. 1B) flow vessels of an exemplary system 102 were equal in diameter and length, but tubes 102 of different diameters and/or lengths from one another may be used as well. As shown in FIGS. 1A and 1B, a first tube 102 is also connected to a left side vessel 112, and a second tube is also connected to a right side vessel 114, so that, for example, fluid can flow from the left side vessel 112 to the first tube 102 to the vessel segment 104 to the second tube 102 and to the right side vessel 114 in a native or forward direction as shown in FIG. 1A and in a backward/reverse direction as shown in FIG. 1B.

The carotid artery segment (an exemplary isolated vessel segment 104) between inserted tubes 102 experienced flow in either forward or reverse directions, and the flow directions were labeled on the vessel in the exemplary experiment, with arrows in FIGS. 1A and 1B showing the direction of fluid flow therethrough. A flow probe 110 (also identified as “F” in FIGS. 1A and 1B) was placed in or in communication with each carotid artery segment 104, such as in the middle of each carotid artery segment 104.

As noted above, two identical silicone tubes 102 (the same diameter and length as the initial two pieces), in at least one embodiment, were inserted into the contralateral artery segment (vessel segment 104) but in different directions to maintain blood flow in the forward direction (i.e., sham). A perivascular ultrasonic flow probe (Transonic Systems Inc., Ithaca, N.Y.) (exemplary flow probe 110) was placed around the middle portion of each vessel segment 104 to monitor carotid flow rate as shown in FIGS. 1A and 1B. FIG. 1C shows blood flow recorded from each carotid artery with an ultrasonic flow probe during the entire experiment. The flow rates from both sides were kept at similar level, but in opposite directions.

For superoxide measurements, carotid arteries (vessel segments 104) from both sides were quickly removed and placed into a heated (37° C.) chamber filled with HEPES buffered PSS containing (in mM): D-glucose, 5.5; KCl, 4.7; NaCl, 142; HEPES sodium salt, 2.7; MgSO₄, 1.17; CaCl₂, 2.79; at pH 7.4. For NADPH oxidase activity assays or protein expression measurements by western blot, the left and right carotid arteries were removed and saved in a −80° C. freezer until analysis.

For actin staining, vessels under different conditions were cut into 4 mm rings and immediately fixed for 2 hours in 4% methanol-free formaldehyde-PBS solution (Thermo Scientific, Rockford, Ill.). For in vitro actin depolymerization immunochemistry, 3 to 4 mm carotid artery ring from control animal were incubated with 10 μM cytochalasin D for 30 minutes. Samples were fixed for 2 hours and then processed for actin staining.

### In Vivo Drug Treatment

In some experiments (n=3), the carotid arteries were treated with jasplakinolide to stablize the actin. After both carotid arteries were exposed and set up as shown in FIGS. 1A and 1B, the left side vessel 112 was first prefilled with 0.1 μM jasplakinolide and incubated for 10 minutes by clamping tubes 104 using clamps 116, 118 as shown in FIGS. 1A and 1B. The right side vessel 114 was filled with saline and served as control. The reverse or forward flows were then ensured for 2 hrs (for superoxide measurement) or 4 hrs (for NADPH oxidase measurment by western blot) after clamps 116, 118 were released. A low concentration of jasplakinolide was used, which was sufficient to stabilize actin and to avoid possible non-specific effects at high concentration. For apocynin treatment, a bolus (4 mg/kg, intravenously) was introduced before initiation of reverse flow followed by continuous infusion (3.5 μg/kg/min, i.v.) during the entire experimental procedure. Accordingly, exemplary in vivo system 100 embodiments of the present disclosure may comprise two tubes 102, a flow probe 110, and optionally one or more clamps 116, 118.

### Superoxide Measurement

The harvested vessels (vessel segments 104) were cut into small rings at approximately 3 mm in length in 37° C. HEPES PSS buffer. The rings were first transferred into 94 well plates with HEPES-PSS and incubated at 37° C. for 5 minutes. Various compounds [cytochalason D, latrunculin B, jasplakinolide, and phorbol 2-myrisate 13-acetate (PMA)] were then added into each well for treatment. The L012 (100 μM) was added last and the luminescence was recorded for 60 minutes at 1 minute intervals using a plate reader from Perkin Elmer (En vision Xcite Multilabel Reader, Perkin Elmer, Shelton, Conn.) at ultra-luminecent mode. The total reaction volume was 200 μl. After each recording, the sample rings were air dried and weighed. Each condition was tested in triplicate. To remove the endothelial cells, the vessel ring was gently rubbed against the tip of a pair of forceps.

Cytochalasins, a class of fungal metabolites, can bind to the growing ends of actin with high affinity and prevent addition of monomers to these sites and have been used as actin depolymerization agent. The toxin latrunculin B sequesters G-actin monomers, and therefore inhibits actin polymerization. Jasplakinolide can bind to and stabilize filamentous actin at low concentrations. Phorbol 12-myristate 13-acetate (PMA) has been shown to be a strong superoxide stimulator by activating protein kinase C and is commonly used as positive control to stimulate superoxide production in different type of cells.

### NADPH Oxidase Activity Assay

Stored samples were placed in ice cold lysis buffer containing 20 mM KH₂PO₄, 1 mM EGTA and protease inhibitor cocktail (Sigma-Aldrich, St. Louis, Mo.) and homogenized with an electric homogenizer (Power Gen 35, Fisher Scientific) for 20 seconds each. The homogenates were centrifuged at 1000 g for 10 minutes at 4° C. to remove the unbroken cells and large tissue debris. The supernatants were collected and kept on ice. The enzyme activity was determined in 200 μl assay buffer at pH7.4 containing the following (in mM): HEPES, 25; NaCl, 120; KCl, 5.9; MgSO₄, 1.2; CaCl₂, 1.75; glucose, 11; β-NADPH, 0.1; and L012, 0.1. The reaction started by adding 10 μL of homogenate, and the luminescence was measured every 15 seconds for 5 minutes by Perkin Elmer plate reader at ultra-sensitive mode. The protein concentration was determined by BCA kit (Bio-Rad) and the results of enzyme activity were standardized to protein level of each sample.

### Immunoprecipitation and Western Blotting for NADPH Oxidase Expression and Phosphorylation

The artery segments were homogenized in the lysis buffer containing 50 mM glycerophosphate, 100 μM sodium orthovanadate, 2 mM magnesium chloride, 1 mM EGTA, 0.5% Triton X-100, 1 mM DL-dithiothreitol, 20 μM pepstatin, 20 μM leupeptin, 0.1 U/ml aprotinin, and 1 mM phenylmethylsulfonyl fluoride and then incubated on ice for 1 hour. The sample was centrifuged at 1000 g for 15 minutes at 1° C., and the supernatant was collected. The total protein was measured by a BCA kit (Bio-Rad). For total NADPH oxidase expressnion, equal amounts of protein (50 μg) from each sample were loaded on each lane and electrophoresed in 4-20% Tris-Glycine gel (Invitrogen) and then transferred onto a polyvinylidene difluoride membrane (Millipore). After being blocked for 2 hours in 6% dried milk in TBS-Tween buffer, the membrane was incubated overnight at 4° C. with specific primary antibody (anti-p47^{phox} and anti-gp91^{phox}, Santa Cruz Biotech) at 1:200 dilution in blocking buffer. The membrane was then rinsed and incubated with horseradish peroxidase-conjugated secondary antibody (Santa Cruz Biotechnology) for 2 hours at a 1:5,000 dilution in blocking buffer. All samples from each group were also probed with anti-β-actin antibody (at a 1:1,000 dilution in blocking buffer, Santa Cruz Biotechnology) to correct for sample loading.

For p47^{phox} phosphorylation measurements, the total protein with phosphoserine was first immunoprecipitated according to a known method. Briefly, samples were first incubated with rabbit polyclonal antibody to phosphoserine (Abcam, Cambridge, Mass.) for 2 hours at 4° C. Samples were then mixed with Protein G PLUS-Agarose (Santa Cruz Biotechnology, Santa Cruz, Calif.) and incubated overnight at 4° C. Samples were then centrifuged and washed three times with lysis buffer. The pellet was resuspended in loading buffer and denatured. The p47^{phox} was then measured by western blot.

### Actin Immunostain

The fixed samples were permeablized by 0.1% tritonX-PBS solution. After non-specific sites were blocked by 1% BSA in PBS, actin was labled by incubating samples with Alexa Fluor488-phalloidin (Invitrogen, Carlsbad, Calif.) for 30 minutes at room temperature. After labeling, each sample was cut into a 3×3 mm piece and mounted on glass slide and covered with a glass cover slip in the presence of 70% glyceral-PBS and sealed with glue. The samples were observed under a Zeiss confocal microscope (Zeiss 510 meta) and images were captured under 100× objective oil lens with excitation at 488 nm and emission at BP 500-530 nm.

### Data Analysis

All data are expressed as mean±SD. Statistical significance was determined using t-test or ANOVA. P<0.05 was considered statistically significant.

### Results

### Actin Depolymerization and Superoxide Production in Endothelial Cells

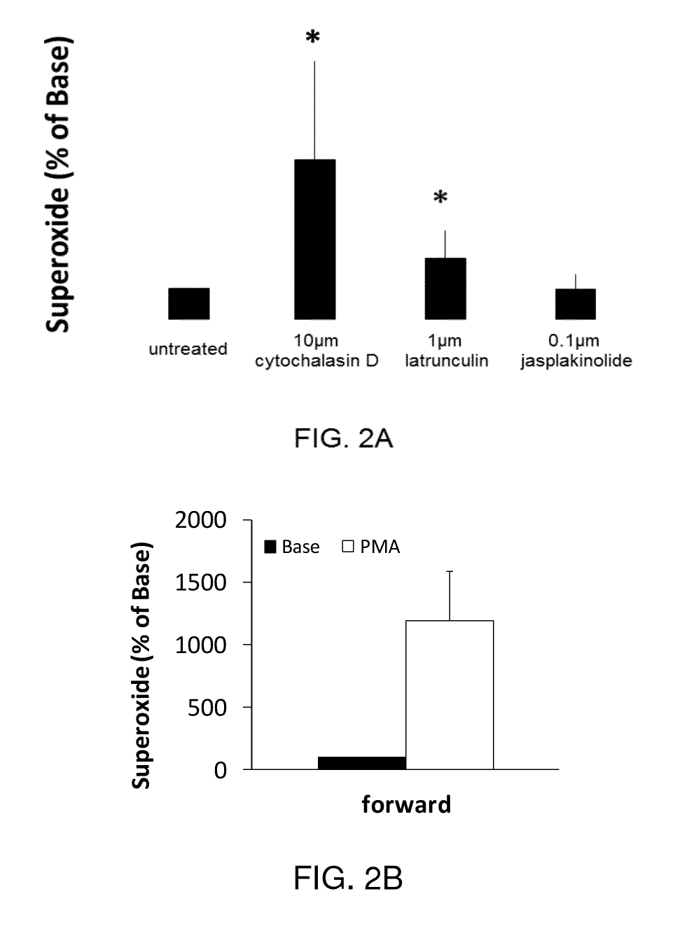

Basal superoxide production was detected in the carotid artery (vessel segment 104) samples obtained from control animals with forward blood flow as shown in FIG. 2A. Rings were incubated with or without treatments of cytochalasin D (cyt D), latrunculin (Latru) or jasplakinolide (Jasp) and the superoxide production were recorded using plate reader at ultrasensitive mode for luminescence to generate the results shown in FIG. 2A. The luminescence recorded from rings without treatment was considered at base level and taken as 100%. The luminescence recorded under other conditions was expressed as percentage of base level. The superoxide production induced by PMA was used as positive control as identified in FIG. 2B. Each condition was tested in triplicate from each animal. The significance of increased superoxide production was test using ANOVA (p=0.004 for cytochalasin D, n=10 and p=0.044 for Latrucillin, n=3), and the data are mean±1SD.

As referenced above, and to determine the role of actin in the regulation of superoxide production, superoxide was measured in vessel segment 104 samples under normal forward blood flow in the presence of either cytochalasin D, latrunculin B or jasplakinolide. The superoxide production in artery sample was increased by about 5 fold with 10 μM cytochalasin D or 2 fold with 1 μM latrunculin treatments (FIG. 2A) as compared to the untreated samples from the same animal. When the artery samples were treated with 0.1 μM jasplakinolide, the superoxide level was not changed, as shown in FIG. 2A.

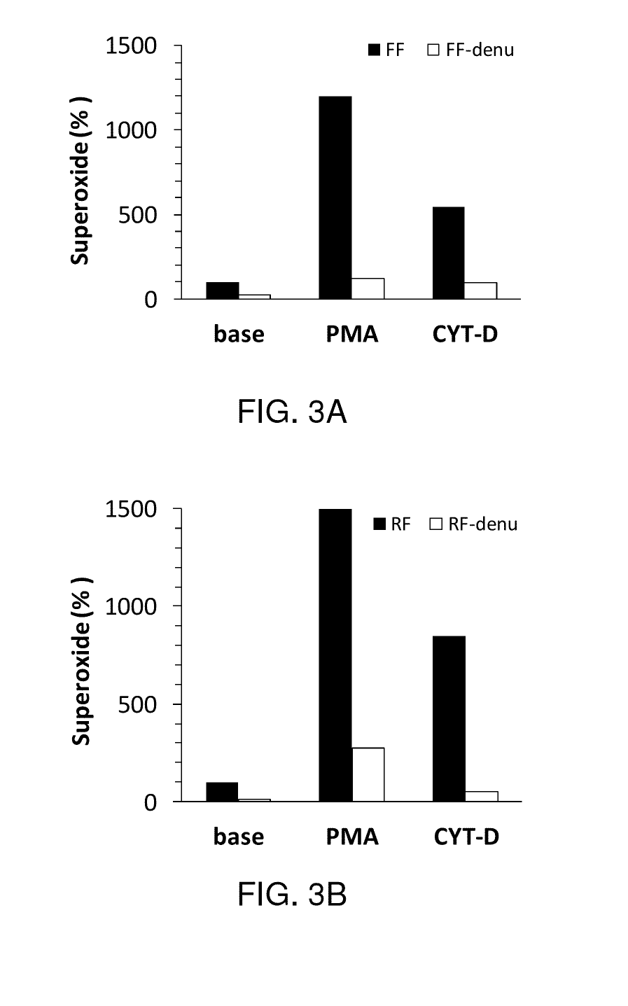

In order to determine the source of the superoxide, the same experiments were repeated in denuded samples. There was only 20% of superoxide production remaining in the denuded samples as shown in FIG. 3A.

As shown therein, the solid bars represent superoxide production from samples with endothelium intact. Superoxide production from intact samples without treatment were taken as 100% control. All other conditions were expressed as percentage of those from intact control samples. Samples were obtained from the carotid artery with either forward (FIG. 3A) or reversed (FIG. 3B) flows (FF or RF, respectively). Each condition was performed in triplicate and the data are the average of two separate experiments.

With the present tests, PMA was used to stimulate artery samples obtained from different conditions to confirm the superoxide producing ability in certain samples as shown in FIG. 3B. The removal of endothelial cells from vessel samples also dramatically reduced PMA-induced superoxide production as shown in FIGS. 3A and 3B.

### Reverse Flow Increased Actin Depolymerization and Superoxide Production in Endothelial Cells

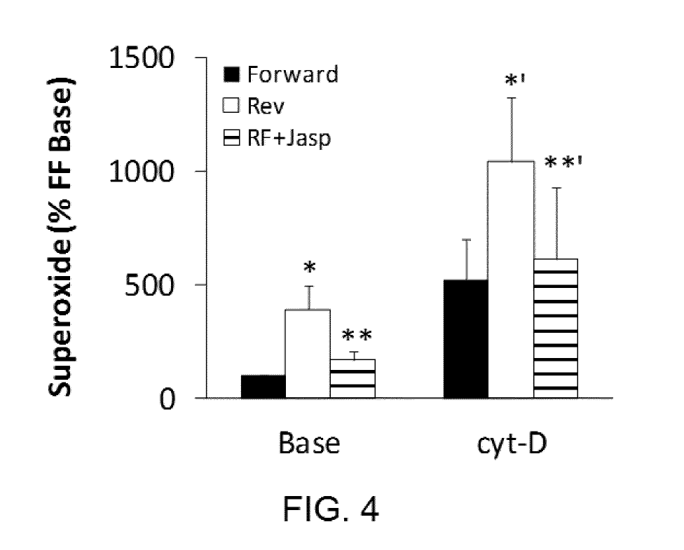

Additional testing was performed to determine if actin was involved in reverse flow-induced superoxide production using the in vivo setup shown in FIGS. 1A and 1B. FIGS. 4A and 4B show the superoxide production in samples from reverse flow vessel increased significantly as compared to the vessel samples with forward flow.

Samples were obtained from carotid arteries with forward (solid bar) or reverse (empty bar) flow (FF or RF, respectively) or reverse flow plus jasplakinolide (Jasp) treatment (lined bar). The basal level of superoxide measured under forward flow condition was taken as 100%, and all other measurements were expressed as percentage to the basal level. Each measurement was performed in triplicate and data were expressed as mean±1SD. A pared t-test was used to examine the significance of difference between superoxide productions from forward and reverse flow samples (*), or between samples with or without Jasp treatment (**) (*p=0.004; *′p=0.04; **p=0.007; **′p=0.003).

Cytochalasin D can further increase superoxide production in vessel with reverse flow. The elevation of superoxide production was greatly reduced in reversed flow samples was greatly reduced after removal of endothelial layer, as shown in FIG. 3B, suggesting that reversed flow induced superoxide production was predominantly from endothelial cells. Apocynin blocked translocation of the NADPH oxidase subunit. When an animal was treated with Apocynin, the reversed flow induced superoxide production was abolished.

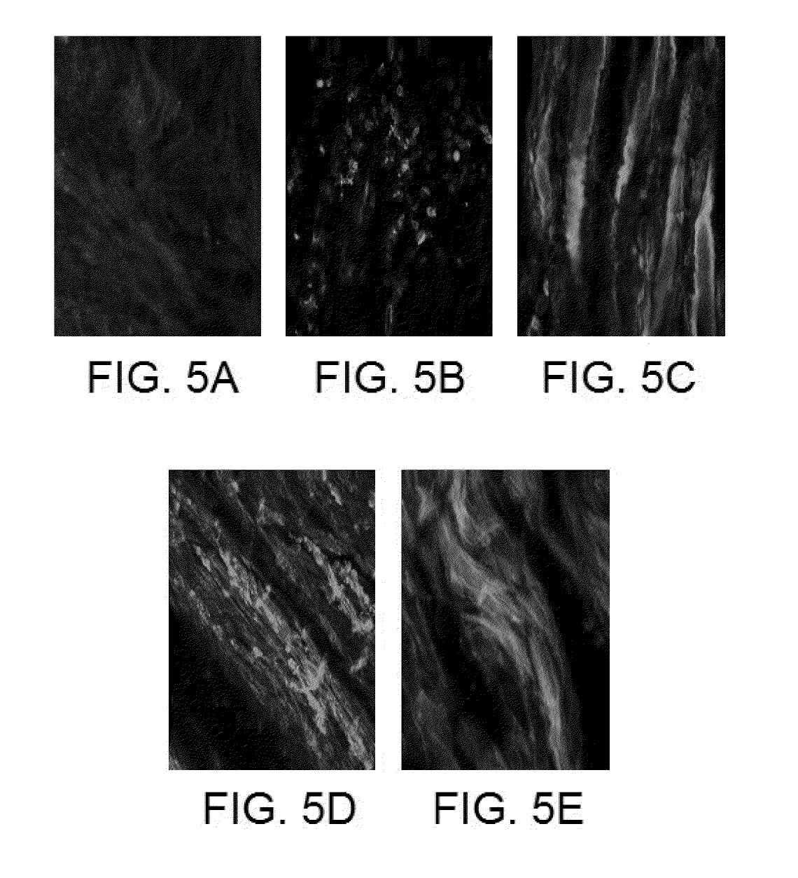

To confirm that reversed flow and the cytochalasin D can inhibit actin polymerization in endothelial cells, the pattern of actin (labeled with fluorescence conjugated pholoidin) in endothelial cells were examined under a confocal microscope. FIGS. 5A-5E show immunostains of actin in carotid artery endothelial cells under forward or reverse flow condition. FIG. 5A shows a negative control, whereby the sample was stained after endothelial layer was removed.

FIG. 5B shows cytochalasin D treatment, whereby the sample was incubated with 10 μM cytochalasin D for 30 minutes at 37° C. before processing. FIG. 5C shows the vessel with forward flow, and FIG. 5D shows the vessel with reverse flow. FIG. 5E shows the vessel with jasplakinolide treatment and reverse flow. All samples were viewed under confocal microscopy (Zeiss 510 meta) with 100× objective with excitation at 488 nm and Emission at BP 500-530 nm.

As shown in the figures, cytochalasin D caused significant disruption of actin filaments (FIG. 5B) which showed dis-arrangement and clamping of actin. Similar findings have been reported previously and the phenomena is concentration dependent. The changes of actin cytoskeleton were visible when cytochalasin D concentration reached 20 nM. When cytochalasin D reached concentration at 2 μM or above, all the long actin filament bundles were disrupted and replaced by large focal aggregates of F-actin. The actin disruption and clamping was also observed in vessels exposed to reverse flow, but to a lesser degree as compared to vessel treated with cytochalasin D (as shown in FIG. 5D). When vessels were treated with jasplakinolide before and during reverse flow, the actin pattern was similar to the vessel with forward flow (FIG. 5E).

### Reverse Flow Increased NADPH Oxidase Activity

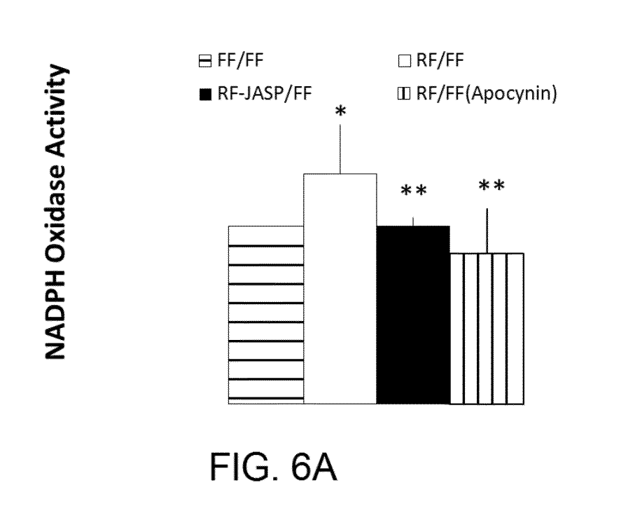

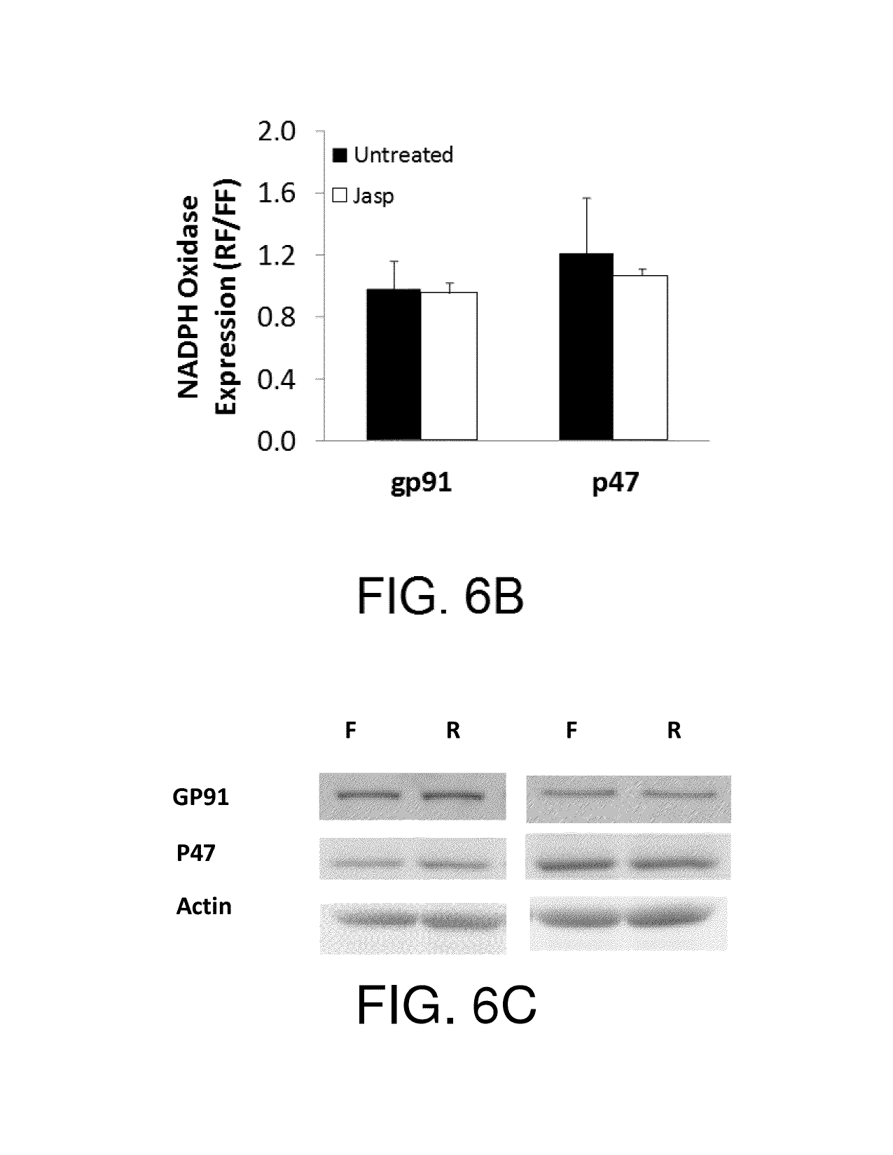

The NADPH oxidase activity was examined using an enzyme assay in subcellular membrane prepared from vessels with different flow conditions. The superoxide produced in samples obtained from vessel with reversed flow was significantly higher than that produced in vessel with forward flow (FIG. 6A), suggesting higher NADPH oxidase activities in samples with reverse flow. FIG. 6A shows the effect of reverse flow on NADPH oxidase activity. The assay was performed in subcellular membrane preparations. The data are expressed as ratio of enzyme activity measured in vessel from reverse flow (RF) or reverse flow with jasplakinolide (RF-Jasp) treatment vs forward flow (FF). The ratio between activities from forward flow samples (FF/FF) is one and served as reference. The ratio of enzyme activity between vessels with forward and reverse flow from Apocynin-treated animal was also plotted and labeled as RF/FF (Apocynin). The data represent means±1SD. FIG. 6B shows the effect of reversed flow on NADPH oxidase protein expression determined by western blot. The data are ratio of protein density between samples with reverse flow and forward flow or reverse flow with Jasp treatment and forward flow. The flow change duration was 4 hours. The data are expressed as ratio of protein band density of reversed flow sample versus that from forward flow sample. FIG. 6C shows the representative images of western blot from samples with forward flow (FF) or reverse flow (RF) (Left, untreated; right, treated with Jasp).

NADPH oxidase activity was decreased in a vessel with reversed flow after jasplakinolide treatment as compared to an untreated vessel. Apocynin blocked translocation of NADPH oxidase subunit, and when the animal was tested with apocynin, the reverse flow-induced superoxide production was abolished.

The level of NADPH oxidase protein was determined by western blot in samples collected from vessel after 4 hours of forward or reverse flow. The protein levels of two major subunits, gp91^{phox} and p47^{phox}, are shown in FIG. 6C. The ratio of protein band density from vessels with reverse flow versus those from forward flow is close to one, suggesting equal levels of protein expressions under these conditions. The expressions of these subunits were also not changed after treatment with jasplakinolide after four hours of treatment.

Reversed Flow and Actin Depolymerization Increased p47^{phox} Phosphorylation

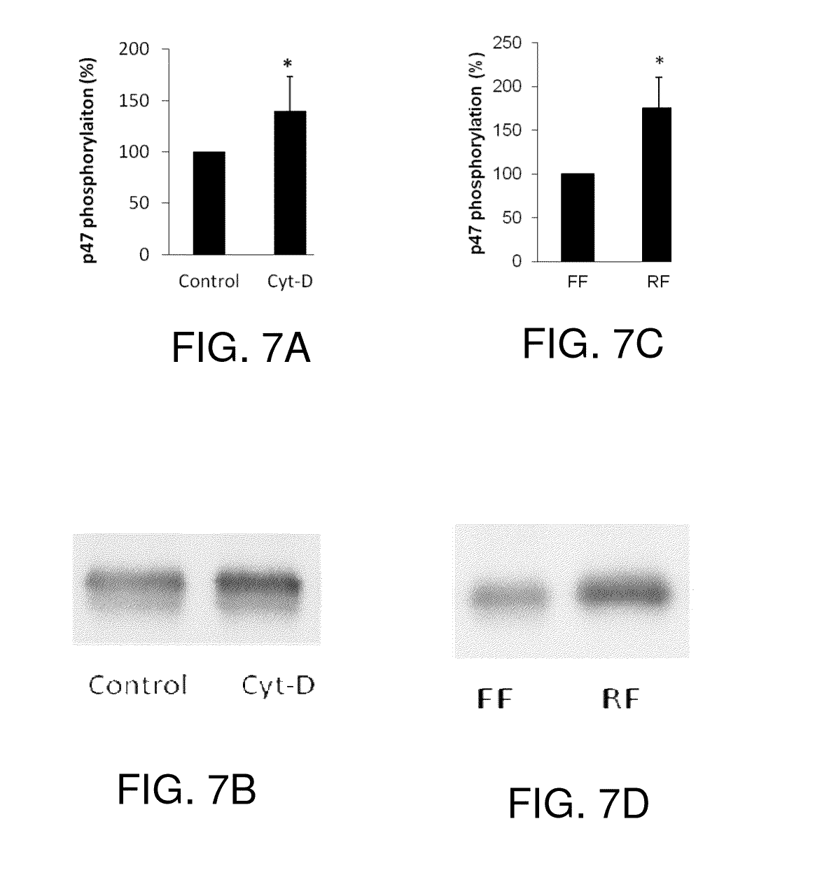

P47^{phox} phosphorylation was examined further to understand the molecular basis for increased superoxide production subsequent to actin depolymerization and flow reversal, as shown in FIGS. 7A-7D. The phosphorylated P47^{phox} was standardized by total P47^{phox}. The level of phosphorylated P47^{phox} at basal level in vitro (FIG. 7A) or under forward flow (FF) condition in vivo (FIG. 7C) were taken as 100% and the level of phosphorylated P47^{phox} after cytochalasin D treatment or under reversed flow (RF) condition were expressed as percent thereof. As shown in FIG. 7A, cytochalasin D treatment induced increased p47^{phox} phosphorylation compared to untreated sample. Under in vivo conditions, the p47^{phox} phosphorylation was also increased in vessels exposed to reversed flow as compared to those with forward flow, as shown in FIG. 7C. The representative images of phosphorylated P47^{phox} are also shown in FIGS. 7B and 7D. The data are mean and SD of at least three experiments from different animals (*p<0.05).

### Discussion

One major finding identified in the present disclosure is that actin mechanical loading plays an important role on the activity of NADPH oxidase system in vascular endothelial cell using both in vitro and in vivo animal models under normal and reverse flow conditions. The results suggest that the actin depolymerization that occurs during reverse flow activates NADPH oxidase (p47^{phox} phosphorylation) and induces superoxide production, and these novel observations may reveal the mechanisms of this mechanical-biochemical interaction.

The regulation of NADPH oxidase has been mostly studied in phagocytes, where disruption of actin polymerization potentiates superoxide production. The phagocyte NADPH oxidase consists of both membrane components (p22, gp91 and Rapl) and cytosolic components (p47 phagocyte oxidase, p67 phagocyte oxidase, Rac, and Rho GDI) or regulatory subunits. Gp91 contains the electron-transporting components of the enzyme, and p22 is a small protein. P67 contains an NADPH binding site and Rac1 or Rac2 is a small GTP protein. P47^{phox} becomes heavily phosphorylated during oxidase activation. When NADPH oxidase is activated, cytosolic components are translocated to the membrane after their phosphorylation by means of various subtypes of protein kinase to enable them to associate with membrane components.

The endothelial NADPH oxidase (Nox) shares many similarities with those of phagocytes. All the classical NADPH oxidase subunits are expressed in endothelial cells. Different NADPH oxidase distribution and regulation have been reported in vascular endothelial and smooth muscle cells. P22^{phox} mRNA and protein can be detected in both endothelial and smooth muscle cells. In contrast, the expression of gp91^{phox} is confined to endothelial cells. The sequence of gp91^{phox} from endothelial cells is the same as that from leukocytes²³. In non-stimulated endothelial cells, a proportion of the NADPH oxidase enzyme exists as a preassembled intracellular complex associated with the cytoskeleton in mainly perinuclear distribution. Endothelial oxidase constitutively generates small amounts of superoxide in non-stimulated cells, which agrees with the detection of basal constitutive production of superoxide in the intact vessel as identified in the present disclosure and shown in FIGS. 2A and 4.

The interaction of actin and NADPH oxidase in endothelial cells, however, is less understood and controversial. Although the regulation of NADPH oxidase has been widely investigated in phagocytes, it is unclear where the mechanisms translate to endothelial cells. Protein kinase C has been implicated in NADPH oxidase regulation in both phagocyte and non-phagocyte. However, cytochalasin D induced superoxide production is protein kinase C independent in phagocyte. The studies described herein showed that cytochalacin D induced superoxide production is protein kinase c dependent. NADPH oxidase regulation may be different depending on the cell type. Many previous studies have focused on NADPH oxidase and actin interaction in non-phagocytes under culture conditions. In these studies, increased depolymerization enhanced ROS production. The actin network in culture cells differs from primary cells where cells in different stages of culture conditions migrate and maintain different surface tensions which require variable actin polymerization and depolymerization rates. Therefore, the results from culture cells may not always translate to in vivo conditions.

One novel finding identified by studies performed in connection with the present disclosure is that actin depolymerization mediates reverse flow induced superoxide production in endothelial cells under in vivo conditions by increasing p47^{phox} phosphorylation. When blood flow was fully reversed, the actin depolymerization was observed in endothelial cells and superoxide production was increased. The increase of superoxide production was prevented by stabilizing actin using an actin stabilizer, jasplakinolide. It is membrane permeable and can easily diffuse into the cells and selectively binds to F-actin. In the studies performed and reported herein, a low concentration of jasplakinolide was used at nanomolar range which has been shown to be an effective dose. Jasplakinolide did not change basal superoxide production (FIG. 2A), suggesting that the change of superoxide production after jasplakinolide pretreatment was not due to cell damage. To support this finding, the studies performed and reported herein also showed that when actin was depolymerized using cytochalasin D, the superoxide production was increased in intact vessel under in vitro condition (FIG. 2A). In phagocyte, cytochalasins have been shown to induce or potentiate superoxide production by NADPH oxidase. To confirm that this was not due to non-specific effects, latrunculin B, another actin polymerization inhibitor, was also shown to active superoxide production in our experiments (FIG. 2A).

Several cytoplasmic components of the NADPH oxidase are associated with the actin cytoskeleton. Moreover, this association is not permanent, but inducible, as suggested by the fact that VEGF stimulation of EC triggers translocation of p47^{phox} to the membrane ruffles, where the most active actin reorganization takes place.

The studies performed in connection with the present disclosure demonstrated that in vivo reverse flow-induced actin depolymerization regulated p47^{phox} serine phosphorylation and facilitated p47^{phox} translocation; i.e., increased NADPH oxidase activity. The NADPH oxidase enzyme assay supports the hypothesis that reverse flow increased the formation of NADPH oxidase complex, as shown in FIG. 6A. The membrane preparation from vessels pretreated with actin stabilizer, jasplakinolide, prevented this elevation. In addition to native jasplakinolide, two additional actin stabilizers, namely Amphidinolide H and Jasplaskinolide V, are also within the scope of the present disclosure as substances, such as jasplakinolide, that can be used to stabilize actin (reduce and/or prevent actin depolymerization) and ultimately inhibit superoxide production, inhibit NADPH oxidase activity, inhibit p47phox phosphorylation, and/or inhibit NADPH oxidase complex formation, when used in a therapeutically effective amount to treat a patient with a cardiovascular condition, such as blood flow reversal, which can result from heart failure, such as congestive heart failure, for example. NADPH oxidase enzyme assay was performed in tissue homogenate after removal of intact cells and mitochondria by centrifugation. The superoxide produced in this assay was derived from pre-formed and activated enzyme complex. As shown by western blot, the protein level in vessels obtained from different conditions is unchanged (FIG. 6B) which suggests that the increased enzyme activity is not due to increased oxidase expression. Apocynin can block translocation of NADPH oxidase subunits. The enzyme activity was also reduced in samples from animals treated with apocynin. Various forms of actin polymer may have different binding affinity and effects on NADPH oxidase. For example, in a cell-free system, the addition of G-actin activated human neutrophil NADPH oxidase, while the addition of F-actin did not enhance oxidase activation. An actin binding site was identified in p47^{phox34}. The present study showed that increased depolymerization of actin facilitated superoxide production (as shown in FIGS. 2A and 4). This may partially be due to increased G-actin around NADPH oxidase and facilitated p47^{phox} phosphorylation and translocation (i.e., up-regulated enzyme activity).

Multiple phosphorylation sites (serines) in p47^{phox} were identified between serine S303 and S379. Based on external stimuli, these sites were phosphorylated by different kinases, including protein kinase C (PKC), protein kinase A (PKA) and mitogen-activated protein kinases (MAPKs). It has been shown that shear stress causes PKC translocation which co-localizes with actin. The change of actin conformation may facilitate the interaction between PKC and p47^{phox}. P47^{phox} can be phosphorylated at different sites under various conditions and therefore the NADPH oxidase activities may be regulated differentially. This may partially explain the variable findings of actin under different conditions. For example, in an arteriole preparation, a forward flow induced superoxide production was diminished when actin was depolymerized. In cultured endothelial cells, arsenic or hyperoxia induced actin depolymerization or reorganization caused increased superoxide production, which was mediated through p47^{phox}.

There are several enzymatic sources of ROS in mammalian cells, including NADPH oxidase system, mitochondrial electron transport chain, xanthine oxidase and cytochrome p450. Among these, the major source in endothelial cells is the NADPH oxidase system. In an exemplary vessel preparation of the present disclosure, the superoxide was primarily derived from endothelial cells. The removal of endothelial layer decreased superoxide production by nearly 80% in all vessels (under various conditions). In support of said findings, others have shown that PMA stimulate ROS production in aortic segment only when the endothelial layer is intact. In the same study, PMA stimulate ROS production in cultured endothelial cells, but not in cultured smooth muscle cells. Previous studies have shown that the major source of superoxide production during full flow reversal is NADPH oxidase in an in vitro blood vessel preparation. When the animal was pretreated with apocynin, the superoxide production induced by reverse flow was dramatically reduced (FIG. 6A).

To date, there is no satisfactory superoxide measurement method for real time direct measurement in vivo. In the studies of the present disclosure, a chemiluminescence probe was used (L-012) for superoxide measurement. Previous studies showed that L-012 was a very sensitive probe for superoxide measurement in either cell free assay, isolated cells and whole tissue. This probe has also been validated in vascular tissue for superoxide measurement. L-012 is more accurate than other chemiluminescence probes due to lack of redox cycling. The measurement with this probe is also more specific to superoxide, since high concentration of H₂O₂ induced only a small increase in L-012 signal. L-012 has also been shown to detect peroxynitrite in cell free solution in the presence of nitric oxide (NO) and hence raises the concern that NO might interfere with the actual superoxide measurement. L-012 also detects peroxynitrite in isolated mitrochondria, but requires much higher concentration of NO. NO can easily diffuses across membranes and reach the intra-mitochondrial space to form peroxynitite. L-012 when used with intact tissue predominantly detects extracellular superoxide. It is likely that the contribution of peroxynitrite in L-012 signal in the studies of the present disclosure is less than that from superoxide.

It is acknowledged that there is a possible contribution of infiltrating leukocytes in the carotid artery preparations since they can produce high level of superoxide when activated. In the in vitro study (identified in FIGS. 2A and 2B herein), the vessels were immediately harvested fresh from control animals and hence unlikely to have significant infiltration of leukocytes. For the in vivo study (identified in FIG. 4), the forward and reverse flow samples were obtained under identical condition and hence should have a normalized inflammatory response. In addition, when the vessel was denudated, the superoxide production was dramatically reduced (as noted in FIG. 3B) which suggests that endothelium was a predominate source. Hence, any contribution of leukocyte in the vessel wall to superoxide production in the experiments referenced herein should be insignificant.

The disclosure of the present application, as noted above, includes studies that demonstrated that actin cytoskeleton plays an important regulatory role in reverse shear stress induced p47^{phox} phosphorylation and NADPH oxidase superoxide production in vascular endothelial cells. Negative shear stress induced actin network re-organization which induced NADPH oxidase complex formation and superoxide production. The results suggest that actin stabilization may be protective in regions of flow reversal in the cardiovascular system, and as such, and as referenced in detail herein, substances useful to stabilize actin (reduce and/or prevent actin depolymerization), inhibit superoxide production, inhibit NADPH oxidase activity, inhibit p47phox phosphorylation, and/or inhibit NADPH oxidase complex formation, can be used in therapeutically effective amounts to treat a patient with a cardiovascular condition, such as blood flow reversal, which can result from congestive heart failure, for example.

While various embodiments of methods of cardiovascular patient treatment using substances sufficient to reduce actin depolymerization have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method of treating a patient, the method comprising the step of administering a therapeutically effective dose of a substance that reduces actin depolymerization within a vasculature of a patient to treat a cardiovascular condition of the patient.

2. The method of claim 1, wherein the substance is selected from the group consisting of jasplakinolide, Jasplaskinolide V, and Amphidinolide H.

3. The method of claim 1, wherein the cardiovascular condition comprises blood flow reversal.

4. The method of claim 3, wherein the therapeutically effective dose of the substance does not inhibit the incidence of blood flow reversal.

5. The method of claim 1, wherein the cardiovascular condition comprises congestive heart failure.

6. The method of claim 1, wherein the therapeutically effective dose is effective to prevent actin depolymerization with the vasculature of the patient.

7. The method of claim 1, wherein the therapeutically effective dose of the substance also inhibits an effect of superoxide within the vasculature of the patient.

8. The method of claim 1, wherein the therapeutically effective dose of the substance also inhibits superoxide production within the vasculature of the patient, wherein the superoxide production is induced due to the cardiovascular condition.

9. The method of claim 1, wherein the therapeutically effective dose of the substance also inhibits superoxide production by endothelial cells within the vasculature of the patient.

10. The method of claim 1, wherein the therapeutically effective dose of the substance also inhibits NADPH oxidase activity within the vasculature of the patient.

11. The method of claim 1, wherein the therapeutically effective dose of the substance also inhibits p47^{phox} phosphorylation within the vasculature of the patient.

12. The method of claim 1, wherein the therapeutically effective dose of the substance also inhibits NADPH oxidase complex formation within the vasculature of the patient.

13. The method of claim 1, wherein the therapeutically effective dose of the substance treats an effect of blood flow reversal within the vasculature of the patient at a location of the blood flow reversal.

14. The method of claim 1, wherein the therapeutically effective dose is less than 1.0 μM of the substance.

15. The method of claim 1, wherein the therapeutically effective dose does not inhibit a naturally-occurring incidence of superoxide production within the patient, but does inhibit superoxide production by endothelial cells in connection with an incidence of blood flow reversal within the vasculature of the patient.

16. A method of treating a patient, the method comprising the step of administering a therapeutically effective dose of a substance selected from the group consisting of jasplakinolide, Jasplaskinolide V, and Amphidinolide H, to a patient to treat a cardiovascular condition of the patient, wherein the cardiovascular condition includes at least an incidence of blood flow reversal within a vasculature of the patient.

17. The method of claim 16, wherein the therapeutically effective dose of the substance also inhibits superoxide production by endothelial cells within the vasculature of the patient.

18. The method of claim 16, wherein the therapeutically effective dose of the substance also inhibits NADPH oxidase activity within the vasculature of the patient.

19. A method of treating a patient, the method comprising the step of introducing a quantity of a substance within a portion of a cardiovascular system, the quantity sufficient to (a) reduce a rate of actin depolymerization resulting from blood flow reversal within the circulatory system, (b) inhibit superoxide production by endothelial cells within the vasculature of the patient, and (c) inhibit NADPH oxidase activity within the vasculature of the patient.

20. The method of claim 19, wherein the substance is selected from the group consisting of jasplakinolide, Jasplaskinolide V, and Amphidinolide H.
